(19) United States
(12) Patent Application Publication
(10) Pub. No.: US 2012/0009223 A1
(43) Pub. Date: Jan. 12, 2012

(54) COLLAGEN-BASED HYDROGELS LOADED WITH ZnO QDs/pDNA COMPLEXES AS CORNEAL SUBSTITUES

(76) Inventors: Liu Wenguang, Tianjin (CN); Liu Guipei, Tianjin (CN); Yang Jianhai, Tianjin (CN); Zhang Peng, Tianjin (CN)

(21) Appl. No.: 12/913,056

(22) Filed: Oct. 27, 2010

(30) Foreign Application Priority Data
May 21, 2010 (CN) 201010178974.0

(51) Int. Cl. A61K 9/00; A61P 27/02; A61K 31/711; B82Y 5/00

(52) U.S. Cl. 424/400; 514/44 R; 977/774

(57) ABSTRACT

This invention relates to a method of fabricating collagen-based hydrogels loaded with ZnO QDs/pDNA complexes as corneal substitutes. Polycation-modified ZnO Quantum Dots were encapsulated into IPN hydrogels by the adsorption effect of freeze-dried hydrogels. The weight ratio of substitutes and ZnO QDs complex is approximately 425:1. And the weight ratio of ZnO QDs/pDNA is 25:1. This kind of corneal substitutes possess favorable biocompatibility. It is able to induce and promote the regeneration of the cornea and it will degrade along with the regeneration of the cornea. The incorporation of the MPDSAH can enhance the stability of corneal substitutes under the existence of collagenase. ZnO QDs used in this invention can condense DNA effectively and ferry DNA into cells successfully. In the process of transfection, the location and distribution of DNA/vector can be tracked by fluorescence in real time. What's more, the convenience of preparation, long term storage and transportation offers a general method to fabricate a promising corneal substitute.

### REFERENCE TO PENDING APPLICATIONS

This application claims priority to Chinese Patent Application No. 201010178974.0, filed May 21, 2010, the contents of which are hereby incorporated by reference.

### REFERENCE TO MICROFICHE APPENDIX

This application is not referenced in any microfiche appendix.

### BACKGROUND OF THE INVENTION

This invention relates to a method of fabricating collagen-based hydrogels loaded with ZnO QDs/pDNA complexes as corneal substitutes. The substitutes exhibit a function of real-time imaging. In addition, it endowed the tissue-engineered scaffolds with a capacity to mediate gene delivery in situ for treating eye diseases.

Keratopathy, a major cause of blindness, has been paid more attention recently. Corneal endothelial cells lack self-repairing function and cannot be replaced. On this premise, corneal allograft has been considered to be one of the most effective methods to cure the corneal disease. However, the worldwide demand for transplantation of corneal allograft far exceeds the supply, and this situation will worsen with an aging population and increased use of corrective laser surgery. Moreover, the potential risk of disease transmission caused by unhealthy donor corneas cannot be ignored. What's worse, the patients always suffer higher rate of postoperative rejection. Thus, the development of corneal allograft substitute is a critical task at present.

The earliest recorded keratoprostheses date back to 1859, when Heusser implanted a glass into a human patient. Up to now, a variety of keratoprostheses were successfully used in clinical trial, such as Boston Keratoprostheses, AlphaCor™ Keratoprostheses, Seoul type Keratoprostheses, Stanford Keratoprostheses, etc. However, these early synthetic materials cannot completely replace corneal tissue because of the poor biocompatibility. With the development of corneal tissue engineering, the researchers have paid more attention to the biomaterials with good biocompatibility and appropriate biodegradability. Naturally derived materials, mainly extracellular matrices (ECMs), have been used to form hydrogels for construction of tissue engineered corneal substitutes. Collagen, accounting for 75% of cornea dry weight, is the most widely used biomacromolecule. Porous collagen sponge and collagen-based hydrogels provide excellent physiological environment for cell adhesion and proliferation.

However, the collagen without crosslinking still has its own shortcomings such as poor mechanical strength and tendency to degradation, which limits its application. In 1999, Griffith et al in Ottawa University incorporated chondroitin sulfate into collagen, which were crosslinked by glutaraldehyde to achieve a kind of substitutes which showed the similar character with the normal cornea in terms of the morphology, histological structure, and transparency. The obtained hydrogels could be sutured in vitro. Also, they found this collagen/glycosaminoglycan substitute could avoid the inflammation and immunoreaction when implanted into the eyes of rabbits. More recently, Griffith et al. fabricated crosslinked collagen hydrogels as corneal substitutes from porcine or bovine collagen using 1-ethyl-3-(3-dimethyl aminopropyl) carbodiimide (EDC)/N-hydroxysuccinimide (NHS) as the coupling agents. The substitutes were optically clear, sufficiently robust to withstand manipulation and suturing, fully biocompatible, and were able to promote regeneration of corneal cells and nerves when implanted into rabbit and pig models.

However, in clinical conditions, collagen was susceptible to rapid biodegradation in some cases with abnormally high collagenase or matrix metalloproteinases concentrations, e.g. keratoconus or corneal alkali burns. To make the substitutes more resistant to in vivo collagenase digestion and to obtain enhanced mechanical properties, methacryloyloxyethyl phosphorylcholine (MPC) was reportedly incorporated into collagen to form an interpenetrating polymer network (IPN) [Liu W, Deng C, McLaughlin C R, et al. Collagen-phosphorylcholine interpenetrating network hydrogels as corneal substitutes. Biomaterials. 2009, 30(8):1551-1559]. Collagen-MPC hydrogels exhibited superior collagenase resistance compared to pristine collagen hydrogels. After one year post-implantation, corneal tissue, tear film, and sensory nerves were regenerated. Nonetheless, MPC is moisture-sensitive and not easy to synthesize and handle. Poly[N-(3-(methacryloylamino)propyl)-N,N-dimethyl-N-(3-sulfopropyl) ammonium hydroxide] (PMPDSAH) is another type of polyzwitterion that has been widely used, due to its excellent biocompatibility and antifouling properties. Moreover, MPDSAH is more chemically stable than MPC and sufficiently available. This makes MPDSAH a favorable alternative to MPC for enhancing collagenase resistance and mechanical properties of collagen-based corneal substitutes.

Additionally, it is essential to trace the degradation process of corneal substitute scaffolds in situ. Designing an appropriate substitute with desired degradability to meet the demands for corneal tissue regeneration is of importance. However, the reported substitutes lack the function of tracing biodegradation in situ. Whereas, the fluorescence emitted by conventional label molecules such as organic fluorescent dye is unstable.

Due to their excellent size-changeable fluorescent character and photochemical stability, quantum dots are widely used in the field of biolabeling and bioimaging. However, the frequently used CdX (X═S, Se, Te) QDs pose a potential risk to biological system, which restrict their practical application. As an alternative, ZnO QDs offer advantages for their biocompatibility and low cytotoxicity. QD-polymer composites integrating advantages of QD and polymer show promising applications in biomedical field. In general, the composites can be fabricated by using various pathways such as exchanging the ligands on the QD surfaces, transferring the QDs into monomer solution via polymerizable surfactants for polymerization, layer-by-layer techniques, reactions between polymers and ligands on the QD surfaces. In this invention, we successfully fabricated polycation-modified ZnO quantum dots with the dual functions of delivering plasmid DNA and labeling cells. ZnO QD/DNA nanocomplexes were encapsulated into the cornea substitute not only for delivering plasmid DNA, but tracking the degradation of collagen scaffolds in situ. This method reported here can become a strategy to construct a ZnO QD-based nonviral vector for delivering endostatin gene to inhibit corneal neovascularization.

### SUMMARY OF THE INVENTION

The object of this invention is to provide a method of fabricating collagen-based hydrogels loaded with ZnO QDs/pDNA complexes as corneal substitutes. The superiority of this corneal substitute involves excellent mechanical properties, optical properties, biocompatibility and collagenase resistance. Collagen-based hydrogels loaded with ZnO QDs/pDNA complexes can be used not only for corneal tissue engineering scaffold, but also for delivering and probing DNA at the same time.

Polycation-modified ZnO quantum dots were encapsulated into IPN hydrogels by the adsorption effect of freeze-dried hydrogels. The weight ratio of substitutes and ZnO QDs complex is approximately 425:1 and the weight ratio of ZnO QDs/pDNA is 25:1.

Collagen-based interpenetrating polymer network (IPN) hydrogels as corneal substitutes were achieved by crosslinking collagen with 1-ethyl-3-(3-dimethyl aminopropyl) carbodiimide (EDC)/N-hydroxysuccinimide (NHS) while the N-(3-(methacryloylamino) propyl)-N,N-dimethyl-N-(3-sulfopropyl) ammonium hydroxide (MPDSAH) network was crosslinked by poly(ethylene glycol) diacrylate (PEGDA) initiated by Irgacure 2959. Component ratio:Coll-NH2:EDC:NHS=1:1:1 (mol/mol), MPDSAH:PEGDA=2:1 (w/w), Irgacure 2959:MPDSAH=0.02:1 (mol/mol). Collagen used is type I porcine atelocollagen. And quantum dots are PMAA-co-PDMAEMA capped ZnO QDs. The molecular structure is depicted as follows:

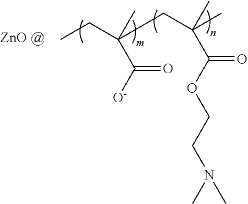

The molar ratio of 2-(N,N-dimethylamino) ethyl methacrylate and zinc methacrylate is 1-30:1

The particle size of ZnO QDs in this invention is approximately 3.6 nm. The concentrations of ZnO QDs and are 5 mg/ml and 0.5 mg/ml, respectively. The thickness and diameter of the corneal substitute are 500-600 nm and 11.5-12 mm respectively.

The corneal substitute provided in this invention can be used in the therapy of ophthalmic related diseases; meanwhile its degradation and in situ release of specific gene can be tracked as well. Compared with the existing products and technique, this invention has many merits: This kind of corneal substitutes possess favorable biocompatibility. It is able to induce and promote the regeneration of the cornea and it will degrade along with the regeneration of the cornea. The incorporation of the MPDSAH can enhance the stability of corneal substitutes under the existence of collagenase. ZnO QDs used in this invention can condense DNA effectively and carry DNA into cells successfully. In the process of transfection, the location and distribution of DNA/vector can be tracked by fluorescence in real time. This kind of corneal substitutes possess multifunctions such as gene transfection, tissue regeneration and fluorescent tracing. What's more, the convenience of preparation, long term storage and transportation offers a general method to fabricate promising corneal substitutes.

Upon reading the included description, various alternative embodiments will become obvious to those skilled in the art. These embodiments are to be considered within the scope and spirit of the subject invention, which is only limited by the claims which follow and their equivalents.

### BRIEF DESCRIPTION OF THE DRAWINGS

FIG. 1 is an image of corneal substitutes under visible light.

FIG. 2 is an image of corneal substitutes without ZnO QDs under ultraviolet

FIG. 3 is an image of collagen/QDs corneal substitutes under ultraviolet

FIG. 4 is an image of IPN1-0.3/QDs corneal substitutes under ultraviolet

FIG. 5 is an image of IPN1-1/QDs corneal substitutes under ultraviolet

FIG. 6 is an image of IPN1-2/QDs corneal substitutes under ultraviolet

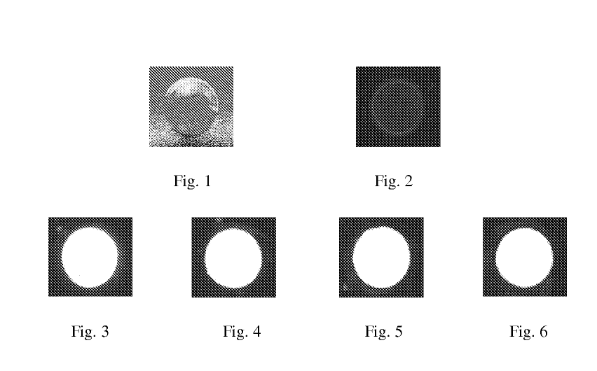

### DESCRIPTION OF THE PREFERRED EMBODIMENTS

The following detailed description shows the best currently contemplated modes of carrying out the invention. The description is not to be taken in a limiting sense, but is made for the purpose of illustrating the general principles of the invention and the best mode for practicing the invention, since the scope of the invention is best defined by the appended claims. The invention is capable of other embodiments and of being practiced or carried out in a variety of ways. It is to be understood that the phraseology and terminology employed herein are for the purpose of description and not of limitation.

According to relevant references [Zhang P, Liu W G. ZnO QD@PMAA-co-PDMAEMA nonviral vector for plasmid DNA delivery and bioimaging. Biomaterials. 2010, 31(11): 3087-3094], water-soluble ZnO quantum dot (ZnO QD)-based nonviral vectors were fabricated by capping the surface of ZnO QD with poly(2-(dimethylamino)ethyl methacrylate) (PDMAEMA), which was synthesized in situ by radical polymerization. This ZnO QD can emit strong yellow luminescence under UV light.

### Example 1

The ratios of Collagen/MPDSAH corneal substitutes are as follows: Type I acidic atelocollagen:MPDSAH=1:0 (w/w), Coll-NH2:EDC:NHS=1:1:1 (mol/mol).

Preparation of corneal substitutes: 0.5 g of 13.7% (w/w) porcine type I acidic atelocollagen solution was transferred into a syringe mixing system. Calculated volumes of 1-ethyl-3-(3-dimethylaminopropyl) carbodiimide (EDC), and N-hydroxysuccinimide (NHS) solution (EDC:NHS:collagen-NH2=1:1:1) were then added to crosslink the collagen and again thoroughly mixed at 4° C. After adjusting the pH to 5.5 using 2N sodium hydroxide, the final mixed solution was immediately dispensed into cornea shaped moulds. The hydrogels were cured at 100% humidity at room temperature for 16 h and then at 37° C. for 5 h. After demoulding, they were washed thoroughly with 20 ml phosphate buffered saline (PBS, pH=7.4), which was replaced at 12 h intervals. The hydrogels were then immersed in PBS containing 1% chloroform to maintain sterility. The corneal substitutes with a thickness of 500-600 μm and a diameter of 11.5-12 mm have the same curvature compared to the human cornea.

Preparation of corneal substitutes loaded ZnO QDs and transfection in vitro: According to the above steps, flat corneal substitutes prepared between two PMMA plates were pumped into pieces with a size of 48-well plates. Then, freeze-dried samples were exposed to ultraviolet light about 24 h for sterilization purposes. ZnO QDs were dissolved in ultrapure water and the solutions were filtered with 0.22 μm sterile filters. Vector/pDNA complexes were then formulated by adding vector of with a concentration (5 mg/ml) to an equal volume of a pDNA solution (0.5 mg/ml). The mixtures were incubated at room temperature for 30 min to allow complex formation. The weight ratio of vector/pDNA was 25:1. The fully sterilized samples were transferred to a 48-well plate containing 500 μl complex solution in each well for 24 h. Aliquots of RCFBF (anterior corneal stroma cells) cell suspension were seeded onto the prepared hydrogel sheets. After incubation at 37° C. in 5% CO2 for 24 h, the medium was then replaced with fresh complete medium and the cells were incubated for an additional 24 h. After the cell lysis, the Luciferase activity was 25957.29, expressed as the number of relative light units (RLU) per mg protein.

Flat hydrogels denoted as Collagen were cut into pieces for mechanical and optical measurements. The rectangular pieces with 20 mm×3 mm×0.50 mm were used for mechanical test.

### Example 2

The components of Collagen/MPDSAH corneal substitutes as follows: Type I acidic atelocollagen:MPDSAH=1:0.3 (w/w), Coll-NH2:EDC:NHS=1:1:1 (mol/mol), MPDSAH:PEGDA (w/w)=2:1, Irgacure 2959:MPDSAH=0.02:1 (mol/mol).

Preparation of corneal substitutes: 0.5 g of 13.7% (w/w) porcine type I acidic atelocollagen solution was transferred into a syringe mixing system, and mixed with calculated volumes of MPDSAH, PEGDA and Irgacure 2959 solution. Mixing was performed in a sealed syringe system immersed in an ice-water bath. The collagen:MPDSAH ratio was 1:0.3 (w/w), MPDSAH:PEGDA ratio was 2:1 (w/w), and Irgacure 2959:MPDSAH ratio was 0.02:1 (mol/mol). Calculated volumes of 1-ethyl-3-(3-dimethylaminopropyl) carbodiimide (EDC), and N-hydroxysuccinimide (NHS) solution (EDC:NHS:collagen-NH2=1:1:1) were then added to crosslink the collagen and again thoroughly mixed at 4° C. After adjusting the pH to 5.5 using 2N sodium hydroxide, the final mixed solution was immediately dispensed into cornea shaped moulds. The moulds were put into a crosslink oven and UV irradiated for about 40 minutes to activate the photo-initiator (Irgacure 2959), and subsequently initiate the copolymerization of MPDSAH and PEGDA. The hydrogels were cured at 100% humidity at room temperature for 16 h and then at 37° C. for 5 h. After demoulding, they were washed thoroughly with 20 ml phosphate buffered saline (PBS, pH=7.4), which was replaced at 12 h intervals. The hydrogels were then immersed in PBS containing 1% chloroform to maintain sterility. The corneal substitutes with a thickness of 500-600 μm and a diameter of 11.5-12 mm have the same curvature compared to the human cornea.

Preparation of corneal substitutes loaded ZnO QDs and transfection in vitro: According to the above steps, flat corneal substitutes prepared between two PMMA plates were pumped into a size of 48-well plates. Then, freeze-dried samples were exposed to ultraviolet light about 24 h for sterilization purposes. ZnO QDs were dissolved in ultrapure water and the solutions were filtered with 0.22 μm sterile filters. Vector/pDNA complexes were then formulated by adding vector of with a concentration 5 mg/ml to an equal volume of a pDNA solution (0.5 mg/ml). The mixtures were incubated at room temperature for 30 min to allow complex formation. The weight ratio of vector/pDNA was 25:1. The fully sterilized samples were transferred to a 48-well plate containing 500 μl complex solution in each well for 24 h. Aliquots of RCFBF cell suspension were seeded onto the prepared hydrogel sheets. After incubation at 37° C. in 5% CO2 for 24 h, the medium was then replaced with fresh complete medium and the cells were incubated for an additional 24 h. After the cell lysis, the Luciferase activity was 25957.29, expressed as the number of relative light units (RLU) per mg protein.

Flat hydrogels denoted as IPN1-0.3 were cut into pieces for mechanical and optical measurements. The rectangular pieces with 20 mm×3 mm×0.50 mm were used for mechanical test.

### Example 3

The components of Collagen/MPDSAH corneal substitutes as follows: Type I acidic atelocollagen:MPDSAH=1:1 (w/w), Coll-NH2:EDC:NHS=1:1:1 (mol/mol), MPDSAH:PEGDA (w/w)=2:1, Irgacure 2959:MPDSAH=0.02:1 (mol/mol).

Preparation of corneal substitutes: 0.5 g of 13.7% (w/w) porcine type I acidic atelocollagen solution was transferred into a syringe mixing system, and mixed with calculated volumes of MPDSAH, PEGDA and Irgacure 2959 solution. Mixing was performed in a sealed syringe system immersed in an ice-water bath. The collagen:MPDSAH ratio was 1:1 (w/w), MPDSAH:PEGDA ratio was 2:1 (w/w), and Irgacure 2959:MPDSAH ratio was 0.02:1 (mol/mol). Calculated volumes of 1-ethyl-3-(3-dimethylaminopropyl) carbodiimide (EDC), and N-hydroxysuccinimide (NHS) solution (EDC:NHS:collagen-NH2=1:1:1) were then added to crosslink the collagen and again thoroughly mixed at 4° C. After adjusting the pH to 5.5 using 2N sodium hydroxide, the final mixed solution was immediately dispensed into cornea shaped moulds. The moulds were put into a crosslink oven and UV irradiated for about 40 minutes to activate the photo-initiator (Irgacure 2959), and subsequently initiate the copolymerization of MPDSAH and PEGDA. The hydrogels were cured at 100% humidity at room temperature for 16 h and then at 37° C. for 5 h. After demoulding, they were washed thoroughly with 20 ml phosphate buffered saline (PBS, pH=7.4), which was replaced at 12 h intervals. The hydrogels were then immersed in PBS containing 1% chloroform to maintain sterility. The corneal substitutes with a thickness of 500-600 μm and a diameter of 11.5-12 mm have the same curvature compared to the human cornea.

Preparation of corneal substitutes loaded ZnO QDs and transfection in vitro: According to the above steps, flat corneal substitutes prepared between two PMMA plates were pumped into a size of 48-well plates. Then, freeze-dried samples were exposed to ultraviolet light about 24 h for sterilization purposes. ZnO QDs were dissolved in ultrapure water and the solutions were filtered with 0.22 μm sterile filters. Vector/pDNA complexes were then formulated by adding vector of with concentration of 5 mg/ml to an equal volume of a pDNA solution (0.5 mg/ml). The mixtures were incubated at room temperature for 30 min to allow complex formation. The weight ratio of vector/pDNA was 25:1. The fully sterilized samples transferred to a 48-well plate containing 500 μl complex solution in each well for 24 h. Aliquots of RCFBF cell suspension were seeded onto the prepared hydrogel sheets. After incubation at 37° C. in 5% CO2 for 24 h, the medium was then replaced with fresh complete medium and the cells were incubated for an additional 24 h. After the cell lysis, the Luciferase activity was 25957.29, expressed as the number of relative light units (RLU) per mg protein.

Flat hydrogels denoted as IPN1-1 were cut into pieces for mechanical and optical measurements. The rectangular pieces with 20 mm×3 mm×0.50 mm were used for mechanical test.

### Example 4

The components of Collagen/MPDSAH corneal substitutes as follows: Type I acidic atelocollagen:MPDSAH=1:2 (w/w), Coll-NH2:EDC:NHS=1:1:1 (mol/mol), MPDSAH:PEGDA (w/w)=2:1, Irgacure 2959:MPDSAH=0.02:1 (mol/mol).

Preparation of corneal substitutes: 0.5 g of 13.7% (w/w) porcine type I acidic atelocollagen solution was transferred into a syringe mixing system, and mixed with calculated volumes of MPDSAH, PEGDA and Irgacure 2959 solution. Mixing was performed in a sealed syringe system immersed in an ice-water bath. The collagen:MPDSAH ratio was 1:2 (w/w), MPDSAH:PEGDA ratio was 2:1 (w/w), and Irgacure 2959:MPDSAH ratio was 0.02:1 (mol/mol). Calculated volumes of 1-ethyl-3-(3-dimethylaminopropyl) carbodiimide (EDC), and N-hydroxysuccinimide (NHS) solution (EDC:NHS:collagen-NH2=1:1:1) were then added to crosslink the collagen and again thoroughly mixed at 4° C. After adjusting the pH to 5.5 using 2N sodium hydroxide, the final mixed solution was immediately dispensed into cornea shaped moulds. The moulds were put into a crosslink oven and UV irradiated for about 40 minutes to activate the photo-initiator (Irgacure 2959), and subsequently initiate the copolymerization of MPDSAH and PEGDA. The hydrogels were cured at 100% humidity at room temperature for 16 h and then at 37° C. for 5 h. After demoulding, they were washed thoroughly with 20 ml phosphate buffered saline (PBS, pH=7.4), which was replaced at 12 h intervals. The hydrogels were then immersed in PBS containing 1% chloroform to maintain sterility. The corneal substitutes with a thickness of 500-600 μm and a diameter of 11.5-12 mm have the same curvature compared to the human cornea.

Preparation of corneal substitutes loaded ZnO QDs and transfection in vitro: According to the above steps, flat corneal substitutes prepared between two PMMA plates were pumped into a size of 48-well plates. Then, freeze-dried samples were exposed to ultraviolet light about 24 h for sterilization purposes. ZnO QDs were dissolved in ultrapure water and the solutions were filtered with 0.22 μm sterile filters. Vector/pDNA complexes were then formulated by adding vector of with a concentration of 5 mg/ml to an equal volume of a pDNA solution (0.5 mg/ml). The mixtures were incubated at room temperature for 30 min to allow complex formation. The weight ratio of vector/pDNA was 25:1. The fully sterilized samples transferred to a 48-well plate containing 500 μl complex solution in each well for 24 h. Aliquots of RCFBF cell suspension were seeded onto the prepared hydrogel sheets. After incubation at 37° C. in 5% CO2 for 24 h, the medium was then replaced with fresh complete medium and the cells were incubated for an additional 24 h. After the cell lysis, the Luciferase activity was 25957.29, expressed as the number of relative light units (RLU) per mg protein.

Flat hydrogels denoted as IPN1-2 were cut into pieces for mechanical and optical measurements. The rectangular pieces with 20 mm×3 mm×0.50 mm were used for mechanical test.

The properties of corneal substitutes from example 1 to example 4 are shown in Table 1 and Table 2

TABLE 1
Optical Properties of Hydrogels

| | White Light Transmission (%) | | |
|---|---|---|---|
| Sample | In PBS | Adsorption of ZnO QDs | Refractive Index^{a} |
| Collagen | 89.6 ± 0.5 | 88.9 ± 0.8 | 1.3511 ± 0.0005 |
| IPN1-0.3 | 87.3 ± 0.8 | 86.6 ± 1.1 | 1.3573 ± 0.0006 |
| IPN1-1 | 89.1 ± 1.3 | 88.5 ± 1.5 | 1.3562 ± 0.0004 |
| IPN1-2 | 87.4 ± 1.4 | 87.3 ± 1.5 | 1.3553 ± 0.0008 |

| | | |
|---|---|---|
| Human cornea | >87 | 1.373-1.380 |

^{a}n = 3

TABLE 2
Mechanical properties of hydrogels

| | Tensile strength (MPa) | | Elongation at break (%) | | Modulus (MPa) | |
|---|---|---|---|---|---|---|
| Sample | bearing ZnO | Without ZnO | bearing ZnO | Without ZnO | bearing ZnO | Without ZnO |
| Collagen | 0.313 ± 0.029 | 0.347 ± 0.044 | 25.29 ± 2.08 | 24.01 ± 4.16 | 1.239 ± 0.101 | 1.477 ± 0.331 |
| IPN1-0.3 | 0.486 ± 0.072 | 0.510 ± 0.089 | 22.27 ± 1.63 | 28.38 ± 7.81 | 2.190 ± 0.350 | 1.848 ± 0.410 |
| IPN1-1 | 0.542 ± 0.102 | 0.535 ± 0.104 | 30.90 ± 10.81 | 30.52 ± 6.96 | 1.814 ± 0.266 | 1.761 ± 0.084 |
| IPN1-2 | 0.563 ± 0.040 | 0.565 ± 0.059 | 31.67 ± 5.16 | 35.03 ± 5.82 | 1.802 ± 0.231 | 1.626 ± 0.113 |

While embodiments of the present invention have been illustrated and described, such disclosures should not be regarded as any limitation of the scope of our invention. The true scope of our invention is defined in the appended claims. Therefore, it is intended that the appended claims shall be construed to include both the preferred embodiment and all such variations and modifications as fall within the spirit and scope of the invention.

We claim:

1. A method of fabricating corneal substitutes loaded with ZnO QDs/pDNA complexes, said method comprising:
encapsulating ZnO QDs into IPN hydrogels by the adsorption effect of freeze-dried hydrogels.

2. The method of claim 1, wherein the weight ratio of said corneal substitutes and said ZnO QDs complex is approximately 425:1 and the weight ratio of said ZnO QDs/pDNA is 25:1.

3. The method of claim 1 wherein said corneal substitutes have a thickness of between 500-600 μm and a diameter of between 11.5-12 mm.

4. The method of claim 1 wherein said corneal substitutes being further defined as:
crosslinking collagen with 1-ethyl-3-(3-dimethyl aminopropyl) carbodiimide (EDC)/N-hydroxysuccinimide (NHS); and
while crosslinking N-(3-(methacryloylamino)propyl)-N,N-dimethyl-N-(3-sulfopropyl) ammonium hydroxide (MPDSAH) network with poly(ethylene glycol) diacrylate (PEGDA) initiated by Irgacure 2959.

5. The method of claim 1 wherein said collagen is type I porcine atelocollagen.

6. The corneal substitutes according to claim 1 being utilized in the therapy of ophthalmic diseases.

7. The method of claim 1 wherein said quantum dots are PMAA-co-PDMAEMA capped ZnO QDs.

8. The method according to claim 7, wherein the particle size of ZnO QDs is approximately 3.6 nm, the concentration of ZnO QDs is approximately 5 mg/ml and DNA is approximately 0.5 mg/ml.

9. The method according to claim 7, wherein the concentration of DNA is 0.5 mg/ml, the weight ratio of said corneal substitutes and said ZnO QDs complex is approximately 425:1, and the weight ratio of said ZnO QDs/pDNA is 25:1.

10. The method of claim 1 being further defined as:
a. dissolving water-soluble collagen in sterile water by stirring at approximately 4° C.;
b. transferring approximately 13.7% (w/w) porcine type I acidic atelocollagen solution into a syringe mixing system;
c. mixing said atelocollagen solution with calculated volumes of MPDSAH, EDC, NHS and Irgacure 2959 solution at 4° C.;
d. adjusting the pH of mixed solution to 5.5 by using approximately 2N sodium hydroxide;
e. dispensing said mixed solution into cornea shaped moulds;
f. placing said moulds into a crosslink oven and UV irradiated for about 40 minutes;
g. curing said solution at 100% humidity at room temperature for 16 h and then at 37° C. for 5 h;
h. demoulding said solution creating hydrogels;
i. washed said hydrogels thoroughly with 20 ml phosphate buffered saline (PBS, pH=7.4);
j. immersing said hydrogels in PBS containing 1% chloroform to maintain sterility;
k. freeze-drying said hydrogels and then transferring said hydrogels to a 500 μl ZnO QDs/pDNA complex solution for 24 h; and
l. freeze-drying said hydrogels to ensure long-term preservation and transportation thereof.
